(19) United States
(12) Patent Application Publication
(10) Pub. No.: US 2024/0100214 A1
(43) Pub. Date: Mar. 28, 2024

(54) SOLID ARTICLE FOR SUSTAINED DELIVERY OF VOLATILE MATERIALS IN INTERIOR SPACE

(71) Applicant: The Procter & Gamble Company, Cincinnati, OH (US)

(72) Inventors: Rahul Vyas, Singapore (SG); Desmond Ng, Singapore (SG); Susana Fernandez Prieto, Benicarlo (ES); Johan Smets, Lubbeek (BE)

(21) Appl. No.: 18/536,468

(22) Filed: Dec. 12, 2023

Related U.S. Application Data
(63) Continuation of application No. PCT/US2023/078263, filed on Oct. 31, 2021.
(60) Provisional application No. 63/424,480, filed on Nov. 10, 2022.

(51) Int. Cl. A61L 9/04

(52) U.S. Cl. CPC A61L 9/048; A61L 2101/46

(57) ABSTRACT

A solid article for sustained delivery of volatile materials in an interior space that is subject to temperature fluctuations of at least 5° C. Such solid article comprises one or more non-aqueous volatile materials in a chemically cross-linked gel. The chemically cross-linked gel is formed by mixing one or more polyols or derivatives thereof with a cross-linking agent at a weight ratio of from 1:0.1 to 1:0.8, and the solid article is characterized by a thermal conductivity of from 0.16 W/mK to 0.2 W/mK.

### FIELD OF THE INVENTION

The present invention relates to a solid article with improved properties for better delivery of volatile materials in an interior space that is potentially subject to significant temperature fluctuations, e.g., at least 5° C., preferably at least 8° C., more preferably at least 10° C.

### BACKGROUND OF THE INVENTION

Devices for dispensing volatile materials into interior spaces are well known. Such devices are typically used to deliver a variety of benefits such as air freshening/scenting and/or malodor removal into enclosed interior spaces, such as those commonly seen in households, commercial establishments (e.g., rooms, closets, toilets, etc.) and vehicles (e.g., cars, trucks, RVs).

For example, air freshening products have been designed for dispensing volatile materials, such as perfume oils. The volatile materials may be contained in membrane-based, wick-based or gel-based delivery systems and dispensed via evaporating the volatile materials from the perfume/scent delivery member, e.g., membrane, wick, or gel. When the temperature of the environment increases significantly, the perfume/scent delivery member in such air freshening products may correspondingly increase in temperature that in turn leads to faster vaporization of the volatile materials therein. This may result in a shorter product life overall (in comparison with a lower temperature in the environment).

Further, the problem of inconsistency in the evaporation rate at different temperatures may be exacerbated by different temperatures in different locations and/or at different times of the enclosed interior space (hereinafter “Touch Points”). For example, the temperature in a smaller interior space (such as a cupboard) may be significantly higher than from that in a bigger interior space (such as a room). For another example, the temperature in a sitting car under the sun may be significantly higher than that of a moving car with AC turned on. Correspondingly, the longevity of the air freshening product may be further shortened due to these High Temperature Touch Points.

Thus, it would be beneficial to provide a perfume/scent delivery member that has improved characteristics for better delivery of an air freshening composition containing volatile materials.

### SUMMARY OF THE INVENTION

The present invention provides a solid article for sustained delivery of volatile materials in an interior space, said solid article comprising one or more non-aqueous volatile materials in a chemically cross-linked gel, wherein said chemically cross-linked gel is formed by mixing one or more polyols or derivatives thereof with a cross-linking agent at a weight ratio of from 1:0.1 to 1:0.8, preferably from 1:0.15 to 1:0.75, more preferably from 1:0.2 to 1:0.75, most preferably from 1:0.25 to 1:0.75; wherein said solid article is characterized by a thermal conductivity of from 0.16 W/mK to 0.2 W/mK, preferably from 0.16 W/mK to 0.19 W/mK, more preferably from 0.16 W/mK to 0.18 W/mK; and wherein said interior space is subject to temperature fluctuations of at least 5° C., preferably from 5° C. to 50° C., more preferably from 8° C. to 30° C., most preferably from 10° C. to 20° C.

The solid article of the present invention is preferably characterized by a Day 1 Weight Loss Ratio of less than 2, preferably less than 1.9, and more preferably less than 1.8, measured according to Test 2 hereinafter.

The one or more non-aqueous volatile materials may be selected from the group consisting of perfumes, deodorizing agents, sanitizing agents, insect repellants, malodor reduction agents, and any mixtures thereof. Preferably, said one or more non-aqueous volatile materials are present in an amount ranging from 3% to 85%, preferably from 15% to 75%, more preferably from 20% to 60% by total weight of the solid article.

The one or more polyols or derivatives thereof may be selected from the group consisting of polyols, polyester polyols, polyglycerols, and any mixtures thereof. Preferably, said one or more polyols or derivatives thereof comprises a polyester polyol, more preferably castor oil.

The cross-linking agent may be selected from the group consisting of isocyanates, isothiocyanates, isocyanurates, polyisocyanates, polyisothiocyanates, and any derivatives thereof. Preferably, said cross-linking agent comprises an aliphatic polyisocyanate, more preferably a bio-based aliphatic polyisocyanate.

The solid article of the present invention may have any form selected from the group consisting of: a tablet, a sheet, a film, and any combinations thereof. Preferably, the solid article is a tablet having a shape selected from the group consisting of: a circular shape, a polygon shape, and a non-circular and non-polygon shape. More preferably, the solid article is a tablet having a non-circular and non-polygon shape with a curved side and/or an open end.

Preferably but not necessarily, the solid article of the present invention has a first central evaporative surface, a second central evaporative surface, and a peripheral evaporative surface between the first and second central evaporative surfaces, while each of the first and second central evaporative surfaces is characterized by an evaporative surface area of greater than 2 cm² and/or less than 200 cm², preferably from 3 cm² to 150 cm², more preferably from 6 cm² to 60 cm².

The present invention also relates to the use of the above-descried solid article for improved delivery of volatile materials in an interior space that is subject to temperature fluctuations of at least 5° C.

### BRIEF DESCRIPTION OF DRAWINGS

FIG. 1 is a perspective view of a solid article for dispensing volatile materials according to the present invention;

FIG. 2 is a front view of a volatile material dispenser with the solid article of FIG. 1; and

FIG. 3 is a perspective view of an air freshening product containing a solid article according to the present invention.

### DETAILED DESCRIPTION OF THE INVENTION

Prior to describing the present invention in detail, the following terms are defined for clarity. Terms not defined should be given their ordinary meaning as understood by a skilled person in the relevant art.

“Solid article” as used herein, refers to a self-supporting, three-dimensional object having a width, length and thickness along an x-axis, y-axis, and z-axis, respectively.

“Interior space” as used herein refers to a finite volume of an enclosed space in a residential, commercial, or vehicle environment. The interior space may include for example rooms in a residential or commercial establishment, such as bedroom, living room, offices, and the like; a sanitary facility such as a bathroom, a toilet, a locker room, and the like; furniture for storage of personal items, including but not limited to shoe cabinets, wardrobes, gym lockers; vehicles, such as cars, trucks, RVs, and the like.

“Substantially free of” as used herein refers to the presence of an ingredient in an amount that is less than 5 wt %, preferably less than 2 wt %, more preferably less than 1 wt %, most preferably not added intentionally and only present as an impurity or reaction byproduct.

“Top notes” as used herein, refer to perfume raw materials having a high volatility.

“Bottom notes” as used herein, refer to perfume raw materials which are less volatile relative to the top notes.

“Volatile material” as used herein, refers to a material that is vaporizable at room temperature and atmospheric pressure without the need of an additional energy source. The volatile composition may be configured for various uses, including but not limited to, air freshening, deodorization, odor elimination, malodor counteraction, pest control, insect control, insect repelling, medicines/medicaments, disinfectants, sanitization, mood enhancement, aromatherapy aid, scented compositions, non-scented compositions, or any other use which requires a volatile material that acts to condition, modify, or otherwise change the atmosphere or the environment. Any suitable volatile material in any amount or form, including a liquid, solid, gel or emulsion, may be used. It should also be understood that when the volatile material is described herein as being “delivered”, “emitted”, or “released”, this refers to the volatization of the volatile component thereof.

It has been found that reliable and sustained delivery of perfumes and other volatile materials by air freshening products, especially despite potential temperature fluctuations at different Touch Points, play an important role in delighting the consumers/users. In particular, it is desirable to control the initial weight loss of volatile materials in air freshening products, especially at High Temperature Touch Points, for improved delivery of volatile materials (e.g., perfumes) into an interior space that are subject to significant temperature fluctuations.

The present invention therefore provides a solid article comprising one or more non-aqueous volatile materials in a chemically cross-linked gel. The chemically cross-linked gel is formed by mixing one or more polyols or derivatives thereof with a cross-linking agent at a weight ratio (i.e., polyol-to-crosslinker weight ratio) of from 1:0.1 to 1:0.8, preferably from 1:0.15 to 1:0.75, more preferably from 1:0.2 to 1:0.75, most preferably from 1:0.25 to 1:0.75. The solid article with the above-described polyol-to-crosslinker weight ratio can be characterized by an optimized thermal conductivity of from 0.16 W/mK to 0.2 W/mK, or from 0.16 W/mK to 0.19 W/mK, or from 0.16 W/mK to 0.18 W/mK. The interior space where the solid article is placed in is subject to temperature fluctuations of at least 5° C., preferably from 5° C. to 50° C., more preferably from 8° C. to 30° C., most preferably from 10° C. to 20° C.

The optimized thermal conductivity of the solid article, which is formed by carefully controlling the polyol-to-crosslinker weight ratio, allows it to function as a perfume/scent delivery member in an air freshening product to ensure effective and sustained delivery of volatile materials. If the thermal conductivity of the solid article is too high (i.e., above 0.2 W/mK), more heat will be transferred faster from surroundings to the solid article each time when it is exposed to a High Temperature Touch Point, thereby speeding up the evaporation of volatile materials from such solid article. Correspondingly, the product longevity may be significantly shortened over time, if the solid article is placed in an interior space with temperature fluctuations and hence repeatedly exposed to High Temperature Touch Points. If the thermal conductivity of the solid article is too low (i.e., below 0.16 W/mK), it may hinder or prevent effective delivery of volatile materials.

The solid article with the above-described improved characteristics may be characterized by a Day 1 Weight Loss Ratio of less than 2, preferably less than 1.9, and more preferably less than 1.8, measured according to Test 2 hereinafter. Day 1 Weight Loss Ratio is reflective of the difference in perfume weight loss between a perfume/scent delivery article placed at a High Temperature Touch Point for 1 day and the same article placed at a Room Temperature Touch Point for 1 day. The lower the Day 1 Weight Loss Ratio, the longer the product longevity. Therefore, the reduced Day 1 Weight Loss Ratio of the solid article may provide an extended product longevity for the air freshening product that such solid article is a part of, especially when such air freshening product is placed in an interior space with significant temperature fluctuations and hence repeatedly exposed to High Temperature Touch Points, without significantly altering the formulation or character of the air freshening composition in the product.

The solid article described hereinafter comprises one or more non-aqueous volatile materials (e.g., perfumes) in a chemically cross-linked gel. Specifically, the chemically cross-linked gel is formed by mixing one or more polyols or derivatives thereof with a cross-linking agent at the above-mentioned weight ratio under suitable conditions to form a cross-linked network with mechanically and thermally stable chemical bonds (e.g., covalent bonds). The one or more non-aqueous volatile materials can be added before, during, or after the cross-linking process, and such volatile materials are thereby immobilized and stabilized by the cross-linked network of such gel for sustained release over time.

For example, the one or more polyols or derivatives thereof, can be mixed with the one or more volatile materials, and optionally a hydroxyl containing polymer is added. Then, the cross-linking agent is added at a temperature from 5° C. to 35° C., preferably from 15° C. to 30° C. and further mixed in order to provide a homogeneous mixture. The mixture is poured into a mould of a desired shape, and then cured, preferably at a curing temperature from 5° C. to 55° C. Such temperatures limit the evaporation of volatile materials. Alternatively, the mixture can be kept at 5° C. or less to delay curing. Curing can then be started at any time by raising the temperature to the curing temperature.

For another example, the one or more polyols or derivatives thereof can be mixed with the cross-linking agent, and optionally a hydroxyl containing polymer, preferably at a temperature from 20° C. to 85° C., more preferably from 30° C. to 75° C., for a duration from 10 mins to 10 hours, preferably from 15 min to 2 hours. The mixture is cooled down, and then the one or more non-aqueous volatile materials are added, preferably at a temperature from 10° C. to 40° C., more preferably 15° C. to 30° C. and can be further mixed, for instance, from 15 mins to 2 hours. The mixture is subsequently poured into a mould of a desired shape and cured, preferably at a curing temperature from 5° C. to 55° C., preferably from 22° C. to 55° C.

Alternatively, all ingredients can be mixed at a low temperature, such as 5° C. or less, and the resulting mixture is then heated to raise its to the curing temperature.

The chemically cross-linked gel so formed may be present at a level of from 15% to 97%, from 30% to 85%, preferably 40% to 80%, by total weight of the solid article. As mentioned hereinabove, the polyol-to-crosslinker weight ratio ranges from 1:0.1 to 1:0.8, preferably from 1:0.15 to 1:0.75, more preferably from 1:0.2 to 1:0.75, most preferably from 1:0.25 to 1:0.75. The one or more non-aqueous volatile materials may be present at a level of from 3% to 85%, preferably from 15% to 70%, more preferably 20% to 60% by total weight of the solid article.

A polyol is a compound containing multiple hydroxyl groups. Suitable polyols or derivatives thereof useful for the practice of the present invention can be selected from the group consisting of: polyols, polyester polyols, polyglycerols, and any mixtures thereof.

Primary alcohols, having terminal hydroxyl groups, typically result in more linear chains. Secondary alcohols without terminal hydroxyl groups are therefore particularly suitable, because they may form a tighter cross-linked gel network with optimized pore sizes and a correlation length of less than 8 nm, preferably from 0.3 nm to 8 nm, more preferably from 0.3 nm to 4 nm. A combination of primary and secondary alcohols is also preferred, since such combination may also result in a tight cross-linked gel network with desired pore sizes and correlation length. Lightly branched polyols and derivatives thereof, such as poly(diethyleneglycol adipates), are preferred for forming more flexible gels.

The polyols or derivatives thereof may have a weight average molecular weight ranging from 60 Da to 10000 Da, preferably from 150 Da to 3000 Da, even more preferably from 500 Da to 2000 Da, even more preferably 600 Da to 1300 Da. Longer polyols and derivatives thereof may result in greater flexibility of the resulting gel.

Diol polyols, having two hydroxyl-functional groups, result after cross-linking in linear polymers or more open networks having large pore size. In contrast, hydroxyl-functional monomers with functionality larger than two form more compact gels with smaller pore sizes. Suitable polyols include: ethylene glycol, propylene glycol, 1,4-butanediol, 1,6-hexanediol, pentaerythritol, 1,2,6-hexanetriol, 4,6-di-tert-butylbenzene-1,2,3-triol, propanetriol (glycerol), 1,2,5-hexanetriol, 1,2,4-cyclohexanetriol, 2,5-dimethylhexane-1,2,6-triol, 3-hydroxymethylpentane-1,2,5-triol, 1,3,6-hexanetriol, 1,1,5,5-pentanetretraol, 1,2,5,6-hexanetretraol, 1,2,3,4,5,6-hexanehexol (sorbitol) and mixtures thereof.

Polyester polyols are hydroxyl-containing esters. Suitable polyester polyols can be selected from the group consisting of: aliphatic polyester polyols, aromatic polyester polyols, organic oil based polyester polyols, and mixtures thereof. Organic oil-based polyester polyols are preferred. Preferred organic oils are vegetable oils since they typically comprise high levels of unsaturation (C═C bonds) and naturally comprise hydroxyl groups. Suitable polyester polyols include: hexanoic acid, 4-hydroxy-, 1,1′,1″-(1,2,3-propanetriyl) ester; pentanoic acid, 5-amino-4-hydroxy-, 1,1′,1″-(1,2,3-propanetriyl) ester; Polycaprolactone triol; castor oil, hydroxyl sunflower oil (HSO) and mixtures thereof.

Castor oil is particularly suitable. Castor oil (Ricinus oil) is a pale yellow and viscous liquid, derived from the bean of the castor plant (Ricinus communis). Castor oil is predominately made up of triglycerides of fatty acids that contain 87-90% of ricinoleic acid (cis-12-hydroxyoctadec-9-enoic acid), and can be achieved in high purity grades. Castor oil and its derivatives have been used as polyols for polyurethanes and adhesives. The castor oil can be partially hydrogenated. It has been found that castor oil provides the length of the branches and the position of the hydroxyl groups which is particularly suited for providing a chemically cross-linked gel having a pore size which results in slow release of the hydrophobic material, particularly where the hydrophobic material is a perfume. In addition, the chemically cross-linked gels derived from castor oil show less syneresis of the hydrophobic material from the gel.

Polyglycerols are hydroxy-containing ethers. Polyglycerols are typically obtained by the polymerisation of alkylene oxides (such as epoxides). Suitable alkylene oxides include ethylene oxide, propylene oxide, butylene oxide, and mixtures thereof, using chain initiators such as ethylene glycol, propylene glycol, diethyelene glycol, dipropylene glycol, 1,4-butanediol, neopentyl glycol, pentaerythritol, hexanetriol, sorbitol, glycerol, and mixtures thereof. Suitable polyglycerols can be selected from the group consisting of: α,α-diglycerol, α,β-diglycerol, hyperbranched polyglycerol, dendritic polyglycerol, and mixtures thereof. Hyperbranched polyglycerols are aliphatic polyethers with multiple hydroxyl end groups that are obtained from the nonsymmetric polyaddition of glycidol to glycerol resulting in a globular branch-on-branch structure which provides special internal flexibility. Dendritic polyglycerols are a hyperbranched polyglycerol with a well-defined symmetric and spherical three-dimensional structure around a core. Apart from improving gel elasticity, the dendritic structure with sterically shielded core together with the exceptionally high number of functional groups of hyperbranched polyglycerols produces flexible gels with relatively low pore size, which increase the longevity of final composition by reducing the diffusion rate not only as a consequence of physically entrapping the hydrophobic material, but also enhancing H-bonding and Van der Waals interactions. Such polyglycerols can be purchased from Nanopartica GmbH (Germany) and Sigma-Aldrich. Suitable polyglycerols include: polyethylene glycol, polypropylene glycol, poly(diethylene glycol), poly(dipropylene glycol), poly(1,4-butanediol), poly(neopentyl glycol), poly(1,6-hexanediol), and mixtures thereof. The polyglycerol preferably has from 2 to 50, preferably from 4 to 30 repeat units.

Any suitable cross-linking agent can be used, although cross-linking agents selected from the group consisting of: isocyanates, isothiocyanates, isocyanurates, oligoisocyanates, polyisocyanates, oligoisothiocyanates, polyisothiocyanates, and any derivatives thereof, are preferred.

The cross-linking agent can be a linear, branched, or cyclic isocyanate, and mixtures thereof. Cyclic isocyanates and mixtures thereof are preferred. Suitable cyclic isocyanates include heterocyclic isocyanates such as 1,3,5-tris(5-isocyanatopentyl)-1,3,5-triazine-2,4,6(1H,3H,5H)-trione.

Suitable cross-linking agents can also be selected from the group consisting of: butane diisocyanate (BDI) such as 1,4-butane diisocyanate, pentamethylene diisocyanate (PDI) such as 1,5-pentamethylene diisocyanate, hexamethylene diisocyanate (HDI) such as 1,6-hexamethylene diisocyanate, xylene diisocyanate (XDI), L-Lysine ethyl ester diisocyanate (LDI), 4,4′-Methylenebis(cyclohexyl isocyanate) (H12MDI), glycolide-ethylene glycol-glycolide isocyanate (Bezwada, LLC), methylene diphenyl diisocyanate (MDI) such as 4,4′-Methylenebis(phenyl isocyanate), 2,4′-Methylenebis(phenyl isocyanate) and 2,2′-Methylenebis(phenyl isocyanate), isophorone diisocyanate (IPDI), toluene diisocyanate (TDI) such as 2,4-toluene diisocyanate and 2,6-toluene diisocyanate, 1,3-bis(2-isocyanatopropan-2-yl)benzene, Poly (pentamethylene diisocyanate), Poly (hexamethylene diisocyanate), 1,3,5-tris(5-isocyanatopentyl)-1,3,5-triazine-2,4,6(1H,3H,5H)-trione, and mixtures thereof.

The cross-linking agent is preferably an oligoisocyanate or polyisocyanate, more preferably a bio-based oligoisocyanate or polyisocyanate. For example, such cross-linking agent can be Desmodur® eco N 7300 commercially available from Sigma-Aldrich and from Covestro (which is a bio-based aliphatic polyisocyanate from PDI), or Stabio™ D-370N commercially available from Mitsui Chemicals (which contains PDI oligomers, isocyanurate (3×)), or Takenate™ D-120N commercially available from Mitsui Chemicals (which is an aliphatic polyisocyanate formed from hydrogenated xylene diisocyanate).

The cross-linking agent may have a viscosity below 2500 mPa S at 25° C. and an isocyanate equivalent weight of from 15% to 40%, preferably from 18% to 30%. Such cross-linking agents are more easily blended with the polyol. As a result, more uniform gels can be achieved.

The resulted chemically cross-linked gel is preferably essentially free, or free of unreacted isocyanates and/or isothiocyanates.

Optionally, the chemically cross-linked gel may further comprise a hydroxyl-containing polymer, a hydroxyl-containing oligomer, or mixtures thereof. The hydroxyl-containing polymer and/or oligomer can be used to alter the elasticity of the gel composition, and therefore the longevity of the perfume release. since a higher elastic modulus G′ slows the perfume release. Suitable hydroxyl containing polymers can be selected from the group consisting of: poloxamers, gelatins, carrageenan, chitin, chitosan, and mixtures thereof.

Poloxamers are nonionic triblock copolymers composed of a central hydrophobic chain of polyoxypropylene (poly(propylene oxide)) flanked by two hydrophilic chains of polyoxyethylene (poly(ethylene oxide)). Suitable poloxamers have a weight average molecular weight of from 1500 g/mol to 15000 g/mol and a poly(ethylene oxide) weight percentage of from 10% to 80%, preferably from 50% to 80%. Suitable poloxamers are commercially available under the tradename of Pluronic® from BASF.

Gelatins are typically translucent, colorless, and typically obtained from collagen from various animal body parts. They are commonly used as a gelling agent in food, pharmaceutical industry, vitamin capsules, photography, and cosmetic manufacturing. Suitable gelatines can have a bloom of from 90 to 300. Bloom is a test to measure the strength of a gel or gelatin, and is measured according to the method outlined by Bloom in U.S. Pat. No. 1,540,979. The test determines the weight in grams needed by a plunger with a diameter of 0.5 inch (12.7 mm) to depress the surface of the gel 4 mm without breaking it, at a temperature of 25° C. The result is expressed in Bloom (grades). It is usually between 30 and 300 Bloom. To perform the Bloom test on gelatin, a 6.67% by weight gelatin solution is kept for 17-18 hours at 10° C. prior to being tested.

Carrageenan are sulfated polysaccharide for instance derived from red algae, commonly known as Irish moss. They are typically composed principally of alpha-D-galactopyranose-4-sulfate units and 3,6-anhydro-alpha-D-galactopyranose units. At least three forms are known, designated, respectively, as “iota”, “kappa” and “lambda” carrageenan which differ in the ratios of the two galactopyranose units and accordingly in their sulfate ester content.

Kappa-carrageenan is the principal component in aqueous extracts from Chondrus crispus and Gigartina stellata. It is lower in sulfate ester content than iota and lambda carrageenan.

Chitosan is typically obtained by deacetylation under alkaline conditions of chitin, which is the second most abundant biopolymer in nature, after cellulose. Chitin can be found as an important constituent of the exoskeleton in animals, especially in crustaceans, molluscs and insects, and it is also the principal polymer in the cell wall of certain fungi. Chitin and chitosan are linear polysaccharides composed of randomly distributed β-(1-4)-linked D-glucosamine (deacetylated unit) and N-acetyl-D-glucosamine (acetylated unit). Chitosan has two types of reactive groups that can be grafted: the free amine groups on the deacetylated units and the hydroxyl groups on the C3 and C6 carbons on acetylated or deacetylated units.

The chitosan of the present invention may have a molecular weight from 10,000 g/mol to 4,000,000 g/mol, preferably from 70,000 g/mol to 1,600,000 g/mol. Suitable chitosan may have a degree of de-acetylation of at least 50%, preferably at least 60%, more preferably at least 70%, even more preferably at least 75%.

The non-aqueous volatile materials used in the present invention may be selected from the group consisting of: perfumes, deodorizing agents, sanitizing agents, insect repellants, malodor reduction agents, and any mixtures thereof. The non-aqueous volatile materials may be present at a level ranging from 3% to 85%, preferably from 15% to 75%, more preferably from 20% to 60% by total weight of the solid article.

The above-described chemically cross-linked gel with non-aqueous volatile materials immobilized and stabilized therein may be moulded into the solid article of the present invention, which, when set, is a three-dimensional, self-supporting object. Such solid article of the present invention may be essentially non-aqueous (for example, it may contain less than 5 wt %, preferably less than 1 wt %, of water), and most preferably it is substantially free of water.

The solid article of the present invention can be transparent or even translucent, and it can have any suitable shape, such as star, circular or pyramidal. The solid article may be colored by adding dye. The solid article of the present invention can be moulded or even 3D printed into a variety of shapes and sizes for integration into air freshening products configured for vehicles, residential interior spaces, commercial interior spaces, a household furniture interior space such as cupboards or lockers, a household appliance interior space. Preferably, the household appliance may be selected from the group consisting of: refrigerators, air conditioners, washing machine, automatic dishwashing machine. The interior space may be a portable consumer product interior environment, preferably the portable consumer product may be bags, luggage or the like.

The solid article of the present invention can be implemented into a volatile material dispenser (such as an air freshening apparatus) for delivering volatile materials (such as perfumes) into an interior space. It is contemplated that the dispenser may be configured for use in a variety of applications to deliver volatile materials to the atmosphere and/or a surface, as long as the volatile materials are released by the apparatus. For the purposes of this disclosure, but without intending to limit the scope of the invention, the dispenser is described as a non-energized device.

For the purposes of illustrating the present invention in detail, the invention is described below as a non-energized volatile material dispenser. However, the volatile material dispenser may be configured for use with an energized device such as, for example, an electrical heating device or a fan. In particular, the volatile material dispenser described is a consumer product, such as an air freshener, for evaporating volatile materials in spaces to deliver a variety of benefits such as air freshening/scenting and/or malodor removal in enclosed spaces such as rooms in household and commercial establishments, or a vehicle passenger compartment space. However, it is contemplated that the dispenser may be configured for use in a variety of applications to deliver volatile materials to the atmosphere and the dispenser may include but is not limited to consumer products, such as, for example air freshening products.

The solid article for delivering one or more non-aqueous volatile materials according to the present invention has one or more evaporative surfaces, preferably a plurality of evaporative surfaces, to allow the volatile materials contained therein to evaporate therefrom. Preferably, the solid article has at least one evaporative surface with surface area of greater than 2 cm² and/or less than 200 cm², preferably from 3 cm² to 150 cm², more preferably from 6 cm² to 60 cm². The evaporative surface area can be measured by creating a 3D model of the gel composition using CAD software, and using the CAD software to calculate the surface area. Any suitable CAD software can be used, such as AutoCad® 2013.

For example, the solid article may have at least a central evaporative surface and a peripheral evaporative surface). For another example, the solid article may have a first central evaporative surface, a second central evaporative surface and a peripheral evaporative surface between the first and second central evaporative surfaces. More preferably, each of the first and second central evaporative surfaces is characterized by an evaporative surface area of greater than 2 cm² and/or less than 200 cm², preferably from 3 cm² to 150 cm², more preferably from 6 cm² to 60 cm².

The solid article may be in a form selected from the group consisting of: a tablet, a sheet, a film and combinations thereof. Preferably, the solid article is a tablet comprising a shape selected from the group consisting of: a circular shape, a polygon shape, and a non-circular and non-polygonal shape (e.g., with a curve and/or an open end).

The solid article of the present invention may be characterized by a volume of greater than 2 cm³ and/or less than 500 cm³, preferably from 5 cm³ to 300 cm³, more preferably from 10 cm³ to 100 cm³.

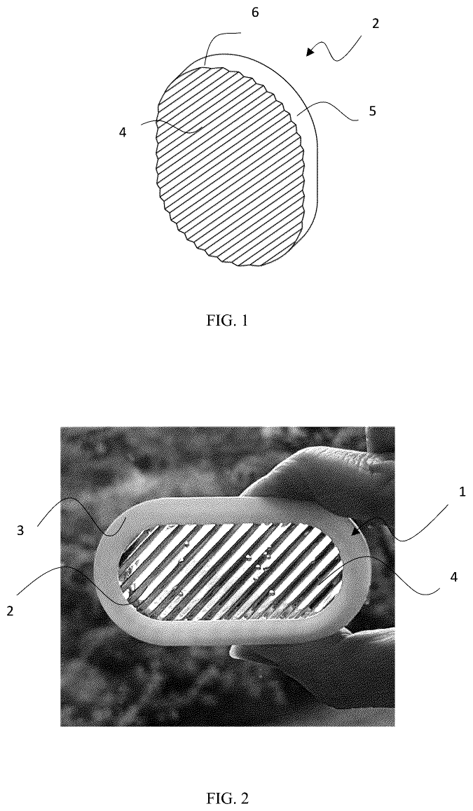

FIG. 1 shows a solid article 2 according to one embodiment of the present invention, which comprises a central evaporative surface 4 and a peripheral evaporative surface 5 surrounding the central evaporative surface 4 defining a thickness of the solid article 2. The peripheral evaporative surface 5 further comprises at least one edge 6 that defines part of an outer periphery of the solid article 2. The solid article 2 may further comprise a second central evaporative surface (not shown in FIG. 1) opposite the central evaporative surface 4. The solid article 2 may comprise a textured surface. In particular, the central evaporative surface 4 comprises an undulating surface profile as shown in FIG. 1.

FIG. 2 shows a volatile material dispenser 1 (e.g., a non-energized air freshening apparatus) according to the present invention, which comprises the solid article 2 of FIG. 1. The volatile material dispenser 1 comprises a housing 3 for housing the solid article 2.

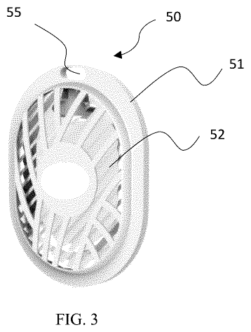

FIG. 3 shows another volatile material dispenser 50 comprising a housing 51 with a solid article 52 according to the present invention. The housing 51 may further comprise a hanging aperture 55 for supporting such dispenser 50 in a vertical orientation above an interior surface. The housing 51 may be made of plastic, paper, or any material chemically compatible with the solid article 52.

### TEST METHODS

### Test 1: Thermal Conductivity Measurement Test Method

Thermal conductivity of the samples described hereinafter are measured by a standard test method ASTM D5930-17 (for Thermal Conductivity of Plastics by Means of a Transient Line-Source Technique) using Xiatech TC3000E transient hotwire thermal conductivity meter Instrument. Measurements were carried out at a temperature of 24±2° C.

### Test 2: Perfume Weight Loss Measurement Method (Day 1 Weight Loss Ratio)

Perfume delivery efficacy of sample solid articles is determined by measuring the weight loss of perfumes (i.e., non-aqueous volatile materials) once the article is exposed to the environment and for a prolonged period of time thereafter. In order to determine the efficacy of a solid article of the present invention in preventing excessive initial weight loss and thereby improving delivery of the perfumes, one might look at the amount of perfumes released on initial use, i.e. Day 1, and determine an initial weight loss ratio.

For calculation of the values detailed herein, one requires the following items:
1. Balance (Scale: Ohaus AA210 S/N 11131122540) or equivalent.
2. Solid article containing perfumes with a pre-determined evaporation surface area.
5. Evaporation rack or equivalent open tray (baker's) rack, covered at the top and shelves spaced at 15 cm or more.
6. Room to accommodate evaporating rack with the following measurements, air flow, temperature/relative humidity or equivalent:
   a) Laboratory Dimensions: 32 feet 4 inches long×72 inches wide×108 inches high or 1,730 ft³
   b) Air Flow (Intake and Exhaust)
      Normal Mode: Average Intake Supply: 103.75 ft³/min±6%
      Average Exhaust: 149.25 ft³/min+6%
      Difference results in negative air pressure: −45.5
      Negative pressure indicates that air supply to laboratory and from an adjacent hallway or room is exhausted through the ventilation system.
   c) Temperature and % Relative Humidity
      Average Temperature: 21° C.+0.1° C. and 35° C.+0.1° C.
      Average % Relative Humidity: 45%±0.5%

### Determination of Day 1 Weight Loss Ratio of Perfume Mixture

1. Measure the weight of the sample at the start
2. Place sample on the evaporation rack in the 21° C. room
3. Measure the weight of the sample after 1 day
4. Repeat steps 1 to 3 with the sample placed in the 35° C. room
5. Calculate 1-day Weight Loss=Weight of the sample after 1 day at the respective temperature (e.g., 21° C. or 35° C.)— Weight of the sample at the start
6. Calculate normalized 1-day weight loss by following equation:

=1-Day weight loss/evaporative surface area of sample
7. Calculate Day 1 Weight Loss Ratio by following equation:

=Normalized 1-Day weight loss at 35° C./Normalized 1-Day weight loss at 21° C.

### EXAMPLES

The following examples are intended to more fully illustrate the present invention and are not to be construed as limitations of the present invention since many variations thereof are possible without departing from the scope of the present invention. All parts, percentages and ratios used herein are expressed as percent weight unless otherwise specified.

### Example I

Inventive solid articles 1 and 2 according to the present invention are prepared based on the composition details described in Table 3 below and according to the process described hereinbefore, which have a thermal conductivity within the claimed range of the present invention. Comparative solid articles A-C with a thermal conductivity outside the claimed range of the present invention are also provided.

TABLE 3

| | | | Thermal |
|---|---|---|---|
| | | | Conductivity |
| Sample | Sample Composition Details | Solid Elastomer | (W/m/K) |
| Inventive | Chemically cross-linked gel formed by | Polyol-to- | 0.163 |
| Sample 1 | mixing polyester polyol (castor oil) | Crosslinker Ratio: | |
| | with polyisocyanate¹ as the | 1:0.72 | |
| | crosslinker, with 50% of perfumes² by | | |
| | total weight | | |
| Inventive | Chemically cross-linked gel formed by | Polyol-to- | 0.175 |
| Sample 2 | mixing polyester polyol (castor oil) | Crosslinker Ratio: | |
| | with polyisocyanate¹ as the | 1:0.27 | |
| | crosslinker, with 20% of perfumes² by | | |
| | total weight | | |
| Comparative | Membrane with liquid phase of | Microporous | 0.224 |
| Sample A | perfume (2.4 ml) | Membrane | |
| | | (Tradename: Teslin ® | |
| | | substrate grade) | |
| | | commercially | |
| | | available from PPG | |
| | | Industries | |
| Comparative | Membrane with liquid phase of perfume | Monolithic | 0.213 |
| Sample B | (6 ml) | Membrane (obtained | |
| | | from Shoshuriki | |
| | | DeOx product by | |
| | | S.T. Corporation) | |
| Comparative | Aqueous gel with solid phase of | Aqueous Gel | 0.557 |
| Example C | perfume (2.5%) with more than 90% | (AmbiPurTM) | |
| | water, and gelling material, stabilizer | | |
| | and emulsifier as remaining | | |
| | composition | | |

¹Stabio D-370N, which contain PDI oligomers formed by a reaction mass of an isocyanurate (a PDI trimer, i.e., 1,5-pentamethylene diisocyanate isocyanurate or 1,3,5-tris(5-isocyanatopentyl)-1,3,5-triazinane-2,4,6-trione) with a trioxotriazine derivative (i.e., (2,4,6-trioxotriazine-1,3,5(2H,4H,6H)-triyl)tris(pentamethylene) isocyanate or 1-{5-[3,5-bis(5-isocyanatopentyl)-2,4,6-trioxo-1,3,5-triazinan-1-yl]penty1}-3,5-bis(5-isocyanatopentyl)-1,3,5-triazinane-2,4,6-trione), as commercially available from Mitsui Chemicals.
²A mixture of perfume raw materials. Perfume compositional details are not provided as any perfumes can be used. Assumptions for the results below are based on the Day 1 Weight Loss Ratio is not dependent on components in the perfume composition.

In order to determine the perfume delivery efficacy, the Day 1 Weight Loss Ratio of Inventive Sample 1 (having a thermal conductivity of less than 0.2 W/mK and even less than 0.18 W/mK) is measured and compared with that of Comparative Samples A-C (having a thermal conductivity greater than 0.2 W/mK), and the results are provided in Table 4 below.

TABLE 4

| | | Normalized | Normalized | |
|---|---|---|---|---|
| | | Day 1 | Day 1 | |
| | | Weight | Weight | |
| | Evaporative | Loss at | Loss at | Day 1 Weight |
| | Surface Area | 21° C. | 35° C. | Loss Ratio |
| Sample | (cm²) | (mg/cm²) | (mg/cm²) | (35° C./21° C.) |
| Inventive | 44 | 9.9 | 17.1 | 1.72 |
| Sample 1 | | | | |
| Inventive | 13 | 5.0 | 9.0 | 1.80 |
| Sample 2 | | | | |
| Comparative | 10 | 4.9 | 11.3 | 2.31 |
| Sample A | | | | |
| Comparative | 33 | 13.5 | 32.6 | 2.42 |
| Sample B | | | | |
| Comparative | 137 | 73.6 | 209.9 | 2.85 |
| Sample C | | | | |

The Day 1 Weight Loss Ratio (35° C./21° C.) is a parameter that can be used to compare to what degree the perfume delivery efficacy of a sample article is affected by the external temperature increase, irrespective of the surface area. A lower ratio indicates that the sample is less affected by the external temperature increase. The Inventive Sample 1 according to the present disclosure have a lower Day 1 Weight Loss Ratios than the Comparative Samples A-C, which can be explained by the lower thermal conductivity of the Inventive Sample 1 as compared to that of the Comparative Samples A-C.

Preferably, the inventive article of the present disclosure is characterized by a Day 1 Weight Loss Ratio of less than about 2, preferably less than about 1.9, and more preferably less than about 1.8.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition as signed to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A solid article for sustained delivery of volatile materials in an interior space, comprising one or more non-aqueous volatile materials in a chemically cross-linked gel; wherein said chemically cross-linked gel is formed by mixing one or more polyols or derivatives thereof with a cross-linking agent at a weight ratio of from 1:0.1 to 1:0.8; wherein said solid article is characterized by a thermal conductivity of from 0.16 W/mK to 0.2 W/mK; and wherein said interior space is subject to temperature fluctuations of at least 5° C.

2. The solid article of claim 1, wherein the weight ratio is from 1:0.15 to 1:0.75, preferably from 1:0.2 to 1:0.75, more preferably from 1:0.25 to 1:0.75; wherein said thermal conductivity is from 0.16 W/mK to 0.19 W/mK, preferably from 0.16 W/mK to 0.18 W/mK; wherein preferably said interior space is subject to temperature fluctuations of from 5° C. to 50° C., preferably from 8° C. to 30° C., more preferably from 10° C. to 20° C.

3. The solid article of claim 1, wherein said solid article is characterized by a Day 1 Weight Loss Ratio of less than 2, preferably less than 1.9, and more preferably less than 1.8.

4. The solid article of claim 1, wherein said one or more non-aqueous volatile materials are selected from the group consisting of perfumes, deodorizing agents, sanitizing agents, insect repellants, malodor reduction agents, and any mixtures thereof; and wherein preferably said one or more non-aqueous volxatile materials are present in an amount ranging from 3% to 85%, more preferably from 15% to 75%, most preferably from 20% to 60% by total weight of the solid article.

5. The solid article of claim 1, wherein said one or more polyols or derivatives thereof are selected from the group consisting of polyols, polyester polyols, polyglycerols, and any mixtures thereof; wherein preferably said one or more polyols or derivatives thereof comprises a polyester polyol, more preferably castor oil.

6. The solid article of claim 1, wherein said cross-linking agent is selected from the group consisting of isocyanates, isothiocyanates, isocyanurates, oligoioscyanates, polyisocyanates, oligoisothiocyanates, polyisothiocyanates, and any derivatives thereof; wherein preferably said cross-linking agent comprises an oligoisocyanate or polyisocyanate, more preferably a bio-based oligoisocyanate or polyisocyanate.

7. The solid article of claim 1, having a form selected from the group consisting of: a tablet, a sheet, a film, and any combinations thereof; wherein preferably the solid article is a tablet having a shape selected from the group consisting of: a circular shape, a polygon shape, and a non-circular and non-polygon shape; wherein more preferably the solid article is a tablet having a non-circular and non-polygon shape with a curved side and/or an open end.

8. The solid article of claim 1, wherein the solid article has a first central evaporative surface, a second central evaporative surface, and a peripheral evaporative surface between the first and second central evaporative surfaces, wherein each of the first and second central evaporative surfaces is characterized by an evaporative surface area of greater than 2 cm² and/or less than 200 cm², preferably from 3 cm² to 150 cm², more preferably from 6 cm² to 60 cm².

9. The use of a solid article for improved delivery of volatile materials in an interior space, said solid article comprising one or more non-aqueous volatile materials in a chemically cross-linked gel; wherein said chemically cross-linked gel is formed by mixing one or more polyols or derivatives thereof with a cross-linking agent at a weight ratio of from 1:0.1 to 1:0.8; wherein said solid article is characterized by a thermal conductivity of from 0.16 W/mK to 0.18 W/mK; and wherein said interior space is subject to temperature fluctuations of at least 5° C.
